(19) United States
(12) Patent Application Publication
(10) Pub. No.: US 2003/0113815 A1
(43) Pub. Date: Jun. 19, 2003

(54) Canine peroxisome proliferator activated receptor gamma

(75) Inventors: Karen L. Houseknecht, Old Saybrook, CT (US); Pamela J. Steele, Niantic, CT (US); Yongling Xiao, Pawcatuck, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/322,332

(22) Filed: Dec. 18, 2002

Related U.S. Application Data
(60) Provisional application No. 60/343,015, filed on Dec. 19, 2001.

(51) Int. Cl. G01N 33/53; G01N 33/567; C12P 21/02; C12N 5/06; C07K 14/705; A61K 31/4439; A61K 31/426

(52) U.S. Cl. 435/7.2; 435/69.1; 435/320.1; 530/350; 514/369; 514/342; 435/350

(57) ABSTRACT

This invention provides polynucleotides encoding canine peroxisome proliferator activated receptor gamma polypeptides, canine peroxisome proliferator activated receptor gamma polypeptides, vectors comprising polynucleotides encoding canine peroxisome proliferator activated receptor gamma polypeptides, transformed cells comprising polynucleotides encoding canine peroxisome proliferator activated receptor gamma polypeptides, antibodies which specifically binds to canine peroxisome proliferator activated receptor gamma polypeptides, microarrays comprising polynucleotides encoding canine peroxisome proliferator activated receptor gamma and therapeutic methods related thereto.

### CROSS-REFERENCE TO RELATED APPLICATION

This non-provisional application claims benefit from U.S. provisional application No. 60/343,015, filed Dec. 19, 2001.

### FIELD OF THE INVENTION

This invention relates to polynucleotides encoding canine peroxisome proliferator activated receptor gamma polypeptides, canine peroxisome proliferator activated receptor gamma polypeptides, vectors comprising polynucleotides encoding canine peroxisome proliferator activated receptor gamma polypeptides, transformed cells comprising polynucleotides encoding canine peroxisome proliferator activated receptor gamma polypeptides, antibodies which specifically binds to canine peroxisome proliferator activated receptor gamma polypeptides, microarrays comprising polynucleotides encoding canine peroxisome proliferator activated receptor gamma and therapeutic methods related thereto.

### BACKGROUND OF THE INVENTION

Peroxisome proliferator activated receptor gamma (PPARγ) is a nuclear hormone receptor that regulates expression of genes that are involved in cell differentiation and lipid metabolism. Nuclear hormone receptors are a family of proteins whose members are both receptors and transcriptional regulators. Nuclear hormone receptors rely on both their receptor function and their transcriptional regulatory function to affect a broad array of biological processes, including development, homeostasis, cell proliferation and cell differentiation (see Mangelsdorf, D. J. et al. (1995) Cell 83:835-840; Wen, D. X., et al. (1995) Curr. Opin. Biotechnol. 6:582-589; Perlmann, T. et al. (1997) Cell 90:391-397; Tenbaum, S. et al. (1997) Int. J. Biochem. Cell Biol. 29:1325-1341; Moras, D. et al. (1998) Curr. Opin. Cell Biol. 10:384-391; Willy, P. J. et al. (1998) in: Hormones and Signaling (ed: B. W. O'Malley) vol. 1, Academic Press, San Diego Calif., pp. 307-358; Weatherman, R. V. et al. (1999) Annu. Rev. Biochem. 68:559-581).

Infestation by fleas and other parasites is common in dogs. Bites from fleas can cause a hypersensitive response known as flea allergic dermatitis (FAD). FAD typically results in localized tissue inflammation and damage, causing substantial discomfort to the afflicted animal. In addition to FAD, dogs are susceptible to dermatitis as a result of food allergies, drug allergies, infection, diabetes and other environmental allergens such as pollen.

U.S. Pat. No. 5,981,586 discloses the use of activators of PPARγ as inhibitors of skin proliferation disease to treat skin diseases.

Dogs are also susceptible to cancer. Cancer cells are characterized by uncontrolled cell proliferation and aberrant cellular metabolism. The induction of terminal differentiation and/or apoptosis has been suggested as a means of slowing down or stopping tumor cell proliferation progression. Demetri, et al., 96 Proc. Natl. Acad. Sci. USA 3951-3956 (1999) report that the administration of the PPARγ agonist, troglitazone, to human patients with advanced liposarcomas results in the in vivo induction of cell differentiation in human solid tumors.

PCT International Application Publication No. WO 96/01317 discloses polynucleotide and polypeptide sequences encoding mouse PPARγ. U.S. Pat. No. 6,200,802 discloses polynucleotide and polypeptide sequences encoding human PPARγ.

### SUMMARY OF THE INVENTION

One aspect of this invention provides isolated polynucleotides encoding a canine PPARγ polypeptide.

Another aspect of this invention provides isolated polynucleotides comprising a polynucleotide sequence that encodes a polypeptide sequence of SEQ ID NO: 2.

An additional aspect of this invention provides isolated polynucleotides comprising a polynucleotide sequence that encodes a polypeptide sequence having at least about 98% amino acid identity with the polypeptide sequence that is encoded by the polynucleotide sequence of SEQ ID NO: 1.

Another aspect of this invention provides isolated polynucleotides characterized as hybridizing under high stringency conditions with a polynucleotide sequence of SEQ ID NO: 1.

A further aspect of this invention provides isolated polynucleotides comprising a polynucleotide sequence having at least about 92% nucleotide identity with the polynucleotide sequence of SEQ ID NO: 1.

An additional aspect of this invention provides isolated canine PPARγ polypeptide.

Another aspect of this invention provides isolated polypeptides comprising a polypeptide sequence having at least about 98% amino acid identity with the polynucleotide sequence that is encoded by the polynucleotide sequence of SEQ ID NO: 1.

A further aspect of this invention provides expression vectors comprising a polynucleotide encoding a canine PPARγ polypeptide.

An additional aspect of this invention provides expression vectors comprising a polynucleotide sequence that encodes a polypeptide sequence of SEQ ID NO: 2.

Another aspect of this invention provides expression vectors comprising a polynucleotide sequence having at least about 92% nucleotide identity with the polynucleotide sequence of SEQ ID NO: 1.

A further aspect of this invention provides transformed cells comprising an inserted polynucleotide comprising a nucleotide sequence that encodes a canine PPARγ polypeptide, or a progeny cell thereof comprising said polynucleotide sequence.

An additional aspect of this invention provides transformed cells comprising an inserted polynucleotide comprising a nucleotide sequence that encodes a polypeptide sequence of SEQ ID NO: 2, or a progeny cell thereof comprising said polynucleotide sequence.

A further aspect of this invention provides transformed cells comprising an inserted polynucleotide comprising a nucleotide sequence having at least about 92% nucleotide identity with the polynucleotide sequence of SEQ ID NO: 1, or a progeny cell thereof comprising said polynucleotide sequence.

Another aspect of this invention provides isolated antibodies which specifically bind to a polypeptide encoded by a sequence of SEQ ID NO: 1.

An additional aspect of this invention provides microarrays comprising an isolated polynucleotide encoding a canine PPARγ polypeptide, preferably, the polynucleotide sequence of SEQ ID NO: 1, or a complimentary nucleotide sequence thereof.

A further aspect of this invention provides microarrays comprising a nucleotide sequence comprising at least 25 contiguous nucleotides of the nucleotide sequence of SEQ ID NO: 1, or a complimentary nucleotide sequence thereof.

Another aspect of this invention provides methods comprising administering a PPARγ activating amount of a PPARγ activator to a mammal of the Canidae family, preferably a member of the species, Canis lupus familiaris, having a disease, condition or disorder which is beneficially treated by an activator of canine PPARγ.

A further aspect of this invention provides methods comprising administering a PPARγ inhibiting amount of a PPARγ inhibitor to a mammal of the Canidae family, preferably a member of the species, Canis lupus familiaris, having a disease, condition or disorder which is beneficially treated by an inhibitor of canine PPARγ.

Another aspect of this invention provides methods comprising administering a PPARγ activating amount of a PPARγ activator to a mammal of the Canidae family, preferably a member of the species, Canis lupus familiaris, having a disease, condition or disorder selected from:

a disease or condition characterized by the cellular expression of TNF-α that is higher than normal;

cancer; and

diabetes mellitus.

In a preferred embodiment of the polynucleotide, expression vector and recombinant cell aspect of this invention said polynucleotide sequence has at least about 95% nucleotide identity with the polynucleotide sequence of SEQ ID NO: 1. In a more preferred embodiment, said polynucleotide sequence is the polynucleotide sequence of SEQ ID NO: 1.

In a preferred embodiment of the expression vector aspects of this invention, the expression vector is as deposited with the ATCC having ATCC deposit number UC-25463.

In a preferred embodiment of the polypeptide aspects of this invention, said polypeptide sequence comprises a polypeptide sequence that is encoded by the polynucleotide sequence of SEQ ID NO: 1.

In a preferred embodiment of the method aspects of this invention, said disease or condition is a disease or condition characterized by the cellular expression of TNF-α that is higher than normal, selected from inflammation, allergy, dermatitis, arthritis, psoriasis, inflammatory bowel disease and endotoxemia.

In another preferred embodiment of the method aspects of this invention, said PPARγ activator is selected from darglitazone, troglitazone, ciglitazone, englitazone, pioglitazone and rosiglitazone.

Unless otherwise defined below, all technical and scientific terms used herein have the same meanings as commonly understood by one of ordinary skill in the art to which this invention belongs.

“Amplification” refers to the production of additional copies of a nucleotide sequence. Amplification is generally carried out using polymerase chain reaction (PCR) technologies are well known in the art.

“Amino acid identity” refers to the sequence alignment of an amino acid sequence calculated against another amino acid sequence, e.g., that of SEQ ID NO: 2. Specifically, the term refers to the percentage of residue matches between two amino acid sequences aligned using a standardized algorithm. Methods of amino acid sequence alignment are well-known. Some alignment methods take into account conservative amino acid substitutions. Such conservative substitutions, explained in more detail above, generally preserve the hydrophobicity and acidity at the site of substitution, thus preserving the structure (and therefore function) of the polypeptide. Preferably percent identity between polypeptide sequences is determined using the default parameters of the CLUSTAL W algorithm as incorporated into the MEGALIGN version 5 sequence alignment program (described and referenced above).

“Hybridization” refers to the process by which a polynucleotide strand anneals with a complementary strand through base pairing under defined hybridization conditions. Specific hybridization is an indication that two nucleic acid sequences share a high degree of identity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml denatured salmon sperm DNA.

Generally, stringency of hybridization is expressed, in part, with reference to the temperature under which the wash step is carried out. Generally, such wash temperatures are selected to be about 5C to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook et al., 1989, Molecular Cloning: A Laboratory Manual, 2^{nd} ed., vol. 1-3, Cold Spring Harbor Press, Plainview, N.Y.; specifically see volume 2, chapter 9.

“High stringency conditions” refers to hybridization between polynucleotides of the present invention which include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, denatured salmon sperm DNA at about 100-200 pg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

“Higher than normal” when referring to the level of expression of TNF-α, particularly with respect to a disease or condition characterized by cellular expression of TNF-α that is higher than normal, means that level of TNF-α expression that is at least 10%, preferably at least 25%, more preferably at least 50% and even more preferably at least 100% higher than normal expression as determined from a population of healthy subjects of the same species, preferably of substantially similar age. The level of TNF-α expression for any particular subject may be determined by methods generally known to those skilled in the art, based upon the present description, including the method described in Example 1 herein.

“Microarray” refers to an arrangement of distinct polynucleotides on a substrate. The terms “element” and “array element” in a microarray context, refer to hybridizable polynucleotides arranged on the surface of a substrate.

“Nucleotide identity” as used herein refers to the sequence alignment of a nucleotide sequence calculated against another nucleotide sequence, e.g. the nucleotide sequence of SEQ ID NO: 1. Specifically, the term refers to the percentage of residue matches between at least two nucleotide sequences aligned using a standardized algorithm. Such an algorithm may insert gaps in the sequences being compared in a standardized and reproducible manner in order to optimize alignment between the sequences, thereby achieving a more meaningful comparison. Percent identity between nucleotide sequences is preferably determined using the default parameters of the CLUSTAL W algorithm as incorporated into the version 5 of the MEGALIGN sequence alignment program. This program is part of the LASERGENE suite of molecular biological analysis programs (DNASTAR, Madison Wis.). CLUSTAL W is described in Thompson, J. D. et al. (1994), Nucleic Acids Research, 22:4673-4680.

“Nucleotide sequence” and “polynucleotide” refer to both DNA or RNA of genomic or synthetic origin which may be single-stranded or double-stranded and may represent a sense or an antisense strand. The term “complimentary nucleotide sequence” refers to a nucleotide sequence that anneals (binds) to a another nucleotide sequence according to the pairing of a guanidine nucleotide (G) with a cytidine nucleotide (C) and adenosine nucleotide (A) with thymidine nucleotide (T), except in RNA where a T is replaced with a uridine nucleotide (U) so that U binds with A.

“Operably linked” refers to a functional relationship between a first nucleotide sequence and a second nucleotide sequence. For example, a promoter sequence is operably linked to a coding sequence if the promoter affects the transcription or expression of the coding sequence. Generally, operably linked DNA sequences may be in close proximity or contiguous and, where necessary to join two protein coding regions, in the same reading frame.

“Recombinant nucleotide sequence” refers to a sequence that is not naturally occurring or that has a sequence that is made by an artificial combination of two or more otherwise separated segments of sequence. This artificial combination is often accomplished by chemical synthesis or, more commonly, by the artificial manipulation of isolated segments of nucleotide sequences, e.g., by genetic engineering techniques such as those described in Sambrook, supra. The term recombinant nucleotide sequence includes a sequence that has been altered solely by addition, substitution, or deletion of a portion thereof. Frequently, a recombinant nucleotide sequence may include a nucleotide sequence operably linked to a promoter sequence. Such a recombinant nucleotide sequence may be part of a vector that is used, for example, to transform a cell.

“Sample” is used in its broadest sense. A sample suspected of containing nucleic acids encoding canine PPARγ, or fragments thereof, or canine PPARγ itself, may comprise a bodily fluid, an extract from a cell, chromosome, organelle, or membrane isolated from a cell, a cell, genomic DNA, RNA, or cDNA, in solution or bound to a substrate, a tissue, a tissue print, etc.

“Specific binding” refers to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide containing the epitope A, or the presence of free unlabeled A, in a reaction containing free labeled A and the antibody will reduce the amount of labeled A that binds to the antibody.

“Substantially purified” refers to nucleotide or amino acid sequences that are removed from their natural environment and are isolated or separated, and are at least 60% free, preferably at least 75% free, and most preferably at least 90% free from other components with which they are naturally associated.

“Substitution” refers to the replacement of one or more amino acids or nucleotides by different amino acids or nucleotides, respectively.

“Transformation” describes a process by which an exogenous nucleotide sequence enters and changes a recipient cell. Transformation may occur under natural or artificial conditions according to various methods well known in the art, and may rely on any known method for the insertion of foreign nucleotide sequences into a prokaryotic or eukaryotic host cell. The method for transformation is selected based on the type of host cell being transformed and may include, but is not limited to viral infection, electroporation, heat shock, lipofection and particle bombardment. The term “transformed cell” includes a stably transformed cells in which the inserted DNA is capable of replication either as an autonomously replicating plasmid or as part of the host chromosome, as well as transiently transformed cells which express the inserted DNA or RNA for limited periods of time.

“Variant” includes “allelic”, “splice”, “or polymorphic” variants. The term is applicable to both nucleotide sequences and amino acid sequences. An “allelic variant” is an alternative form of a nucleotide sequence, for example an alternative form of the sequence encoding canine PPARγ. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence. A “splice variant” may have significant identity to a reference molecule, but will generally have a greater or lesser number of polynucleotides due to alternate splicing of exons during mRNA processing. The corresponding polypeptide may possess additional functional domains or lack domains that are present in the reference molecule. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may, for example, be indicative of a particular population, a disease state or a propensity for a disease state.

The amino acid sequences disclosed herein are written from left to right in the direction from the amino to the carboxy terminus. In accordance with standard nomenclature, amino acid residue sequences are denominated by either a single letter or a three letter code as indicated in Table 1 below:

TABLE 1

| Amino Acid | | |
|---|---|---|
| Residue | 3-Letter Code | 1-Letter Code |
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### BRIEF DESCRIPTION OF THE DRAWINGS

FIGS. 1A and 1B are western blots showing the expression of PPARγ in canine cancer cells, as more fully described in the Detailed Description. FIG. 1A also shows the expression of PPARγ in canine adipose tissue.

FIGS. 2A and 2B illustrate the effect of the PPARγ agonist, darglitazone, on the expression of TNF-α in canine cells, as more fully described in the Detailed Description.

### DETAILED DESCRIPTION OF THE INVENTION

The invention is based in part on a canine peroxisome proliferator activated receptor gamma (PPARγ) and nucleotide sequences encoding the receptor. The invention encompasses said nucleotide sequences, the amino acid sequences of said canine PPARγ and variants thereof. It will be appreciated by those skilled in the art that as a result of the degeneracy of the genetic code, a multitude of nucleotide sequences encoding canine PPARγ, some bearing minimal similarity to the nucleotide sequences of any known and naturally occurring gene, may be produced. Thus, the invention contemplates all variants of the canine PPARγ nucleotide sequences based on all possible codon choices. These combinations are made in accordance with the standard triplet genetic code as applied to the polynucleotide sequence of naturally occurring canine PPARγ, and all such variations are to be considered as being specifically disclosed.

Although nucleotide sequences which encode canine PPARγ and its variants are generally capable of hybridizing to the nucleotide sequence of the naturally occurring canine PPARγ under appropriately selected conditions of stringency, it may be advantageous to produce nucleotide sequences encoding canine PPARγ or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding canine PPARγ and its derivatives without altering the encoded amino acid sequences include the production of RNA transcripts having more desirable properties, such as a greater half-life, than transcripts produced from the naturally occurring sequence. All such coding sequence variants are encompassed by this invention.

Also encompassed by the invention are polynucleotide sequences that are capable of hybridizing to the claimed polynucleotide sequences, particularly to the nucleotide sequence of SEQ ID NO: 1, and fragments thereof, under various conditions of stringency (see, e.g., Wahl, G. M. and S. L. Berger (1987) Methods Enzymol. 152:399-407; Kimmel, A. R. (1987) Methods Enzymol. 152:507-511).

Methods for DNA sequencing are well known in the art and may be used to practice any of the embodiments of the invention. The methods may employ such enzymes as the Klenow fragment of DNA polymerase 1, SEQUENASE (US Biochemical, Cleveland, Ohio), Taq polymerase (Perkin-Elmer, Wellesley, Mass.), thermostable T7 polymerase (Amersham Pharmacia Biotech, Piscataway, N.J.), or combinations of polymerases and proofreading exonucleases such as those found in the ELONGASE amplification system (Life Technologies, Gaithersburg, Md.). Preferably, sequence preparation is automated with machines such as the MICROLAB 2200 liquid transfer system (Hamilton, Reno, Nev.), PTC200 thermal cycler (MJ Research, Watertown, Mass.) and ABI CATALYST 800 thermal cycler (Perkin-Elmer). Sequencing is then carried out using either the ABI 373 or 377 DNA sequencing system (Perkin-Elmer), the MEGABACE 1000 DNA sequencing system (Molecular Dynamics, Sunnyvale, Calif.), or other systems known in the art. The resulting sequences can be analyzed, for example,-using a variety of algorithms which are well known in the art (see, e.g., Ausubel, F. M. (1997) Short Protocols in Molecular Biology, John Wiley & Sons, New York, N.Y., unit 7.7; Meyers, R. A. (1995) Molecular Biology and Biotechnology, Wiley VCH, New York, N.Y., pp. 856-853).

The nucleotide sequences encoding canine PPARγ may be extended utilizing a partial nucleotide sequence and employing various polymerase chain reaction (PCR) based methods known in the art to detect upstream sequences, such as promoters and regulatory elements. For example, one method, restriction-site PCR, uses universal and nested primers to amplify unknown sequence from genomic DNA within a cloning vector (see, e.g., Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (see, e.g., Triglia, T. et al. (1988) Nucleic Acids Res. 16:8186). A third method, capture PCR, involves PCR amplification of DNA fragments adjacent to known sequences in human and yeast artificial chromosome DNA (see, e.g., Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto, Calif.) to walk genomic DNA. This procedure avoids the need to screen libraries and is useful in finding intron/exon junctions. For all PCR-based methods, primers may be designed using commercially available software, such as OLIGO 4.06 Primer Analysis software (National Biosciences, Plymouth, Minn.) or another appropriate program.

When screening for full-length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for the extension of a sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Perkin-Elmer), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences, or fragments thereof, which encode canine PPARγ may be cloned in vectors that direct expression of canine PPARγ, or fragments or functional equivalents thereof, in appropriate host cells. Due to the inherent degeneracy of the genetic code, other DNA sequences which encode substantially the same or a functionally equivalent amino acid sequence may be produced and used to express canine PPARγ.

The nucleotide sequences of the present invention can be engineered using methods generally known in the art in order to alter canine PPARγ-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

This invention encompasses nucleotide sequences which encode canine PPARγ polypeptides and canine PPARγ polypeptide derivatives, or fragments thereof, which are prepared entirely by synthetic chemistry. After production, the synthetic sequence may be inserted into any of the many available expression vectors and cell systems using reagents well known in the art. Moreover, it may be desirable to use synthetic chemistry in order to introduce mutations into a sequence encoding canine PPARγ polypeptides, or fragments thereof.

Sequences encoding canine PPARγ may be synthesized, in whole or in part, using chemical methods well known in the art (see, e.g., Caruthers, M. H. et al. (1980) Nucleic Acids Symp. Ser. 7:215-223 and Horn, T. et al. (1980) Nucleic Acids Symp. Ser. 7:225-232). Alternatively, a canine PPARγ polypeptide or a fragment thereof may be synthesized using chemical methods. For example, peptide synthesis can be performed using various solid-phase techniques (see, e.g., Roberge, J. Y. et al. (1995) Science 269:202-204). Automated synthesis may be achieved using the ABI 431A peptide synthesizer (Perkin-Elmer). Additionally, the amino acid sequence of canine PPARγ, or any part thereof, may be altered during direct synthesis and/or combined with sequences from other proteins, or any part thereof, to produce a variant polypeptide (i.e., a fusion or chimeric protein).

Peptides may be substantially purified by preparative high performance liquid chromatography (see, e.g., Chiez, R. M. and F. Z. Regnier (1990) Methods Enzymol. 182:392-421). The composition of synthetic peptides may be confirmed by amino acid analysis or by sequencing (see, e.g., Creighton, T. (1984) Proteins, Structures and Molecular Properties, WH Freeman, New York, N.Y.).

To express a biologically active canine PPARγ, the nucleotide sequences encoding canine PPARγ, or derivatives thereof, may be inserted into an appropriate expression vector, e.g., a vector containing the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding canine PPARγ. As those skilled in the art will appreciate, such elements may vary in their strength and specificity. Specific initiation signals may also be used to achieve more efficient translation of sequences encoding canine PPARγ. Such signals include the ATG initiation codon and adjacent sequences, e.g. the Kozak sequence. In cases where sequences encoding canine PPARγ and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (see, e.g., Scharf, D. et al. (1994) Results Probl. Cell Differ. 20:125-162).

An expression vector of this invention was prepared by cloning a 1.5 Kb canine PPARγ complimentary DNA (cDNA) into the eukaryotic expression vector pcDNA 3.1/v5/His-TOPO® using the Eukaryotic TOPO TA® Cloning Kit (Invitrogen, Carlsbad, Calif.) according to methods well known to those skilled in the art, including the methods described in the literature published by Invitrogen with respect to pcDNA 3.1/v5/His-TOPO®, and based on the present disclosure. This expression vector, identified as UC-25463, was deposited with the American Type Culture Collection (ATCC, Manassas, Va.) under the terms of the Budapest Treaty on Jun. 13, 2001 and assigned ATCC designation PTA-3454. Hence, it is especially preferred that the expression vector of the present invention is ATCC No. PTA-3454. All restrictions on the availability to the public of the expression vector so deposited will be irrevocably removed upon the issuance of a patent from the specification of the present invention.

Preferably, an expression vector of this invention comprises a polynucleotide encoding canine PPARγ that is operatively linked to a promoter region and/or a promoter region that is operatively linked to an enhancer region (i.e., an enhancer-promoter). Promoters and enhancer-promoters are regions of a DNA molecule that can drive expression of a target peptide in a host cell. The pcDNA 3.1/v5/His-TOPO® vector contains the human cytomegalovirus (CMV) promoter-enhancer. However, other promoters and promoter-enhancers are well known to those skilled in the art and may be used in the invention. In an embodiment, the expression vector comprises a coding sequence useful in the purification of the expressed peptide. For example, the pcDNA 3.1/v5/His-TOPO® vector includes a polyhistidine tag sequence that may be used to purify an expressed peptide using a nickel column. In another embodiment, the expression vector comprises coding sequences that afford a method of selecting successfully transfected host cells from those without the vector. As those skilled in the art will understand, exemplary selection sequences are genes that convey antibiotic resistance to the transfected host cell.

Methods which are well known to those skilled in the art may be used to construct expression vectors containing sequences encoding canine PPARγ and appropriate transcriptional and translational control elements. These methods include in vitro recombinant DNA techniques, synthetic techniques, and in vivo genetic recombination (see, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York, N.Y., ch. 9, 13, and 16).

As those skilled in the art will appreciate, based upon the present description, any suitable expression vector/host systems may be utilized to contain and express sequences encoding canine PPARγ. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. The invention is not limited by the host cell employed.

Those skilled in the art will appreciate, based upon the present description, that in bacterial host systems a number of cloning and expression vectors may be selected depending upon the use intended for polynucleotide sequences encoding canine PPARγ. For example, routine cloning, subcloning, and propagation of nucleotide sequences encoding canine PPARγ can be achieved using a multifunctional E. coli vector such as PBLUESCRIPT™ (Stratagene, La Jolla, Calif.) or PSPORT1 plasmid (Life Technologies). Ligation of sequences encoding canine PPARγ into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (see, e.g., Van Heeke, G. and S. M. Schuster (1989) J. Biol. Chem. 264:5503-5509). When large quantities of canine PPARγ are needed, e.g. for the production of antibodies, vectors which direct high level expression of canine PPARγ may be used. For example, any suitable vector containing the strong and inducible T5 or T7 bacteriophage promoter may be used.

Yeast expression systems may be used for production of canine PPARγ. A number of vectors containing constitutive or inducible promoters, such as alpha factor, alcohol oxidase, and PGH promoters, may be used in the yeast Saccharomyces cerevisiae or Pichia pastoris. Such vectors can direct either the secretion or intracellular retention of expressed proteins and enable integration of foreign sequences into the host genome for stable propagation (see, e.g., Ausubel, 1995, supra; Bitter, G. A. et al. (1987) Methods in Enzymology 153, 516-544; and Scorer, C. A. et al. (1994) Biotech. Adv. 12,181-184).

As those skilled in the art will appreciate, based upon the present description, any suitable plant systems may be used for the expression of canine PPARγ polypeptides of the invention. Transcription of nucleotide sequences encoding canine PPARγ may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (see, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105). As those skilled in the art will appreciate, these constructs can be introduced into plant cells by direct DNA transformation or pathogen-mediated transfection (see, e.g., The McGraw Hill Yearbook of Science and Technology (1992) McGraw Hill, New York, N.Y., pp.191-196).

In mammalian cells, a number of viral-based expression systems may be utilized. For example, in cases where an adenovirus is used as an expression vector, a sequence encoding canine PPARγ may be ligated into an adenovirus transcription/translation complex consisting of the late promoter and tripartite leader sequence. Insertion in a non-essential E1 or E3 region of the viral genome may be used to obtain infective viruses which express canine PPARγ polypeptides in host cells (see, e.g., Logan, J., et al. (1984) Proc. Natl. Acad. Sci. USA 81, 3655-3659). In addition, transcription enhancers, such as the Rous sarcoma virus (RSV) enhancer, may be used to increase expression in mammalian host cells. Simian Virus 40 (SV40) or Epstein Bar Virus (EBV) based vectors may also be used for high-level protein expression.

In an embodiment of the invention, human artificial chromosomes (HACS) may also be employed to deliver larger fragments of DNA than can be contained in and expressed from a plasmid. For example, HACs of about 6 kb to 10 Mb may be constructed and delivered via conventional delivery methods (liposomes, polycationic amino polymers, or vesicles) for therapeutic purposes (see, e.g., Harrington, J. J. et al. (1997) Nat. Genet. 15:345-355).

In another embodiment of the invention wherein long term production of recombinant proteins in mammalian systems is desired, stable expression of canine PPARγ in cell lines is preferred. Methods and techniques for preparing and utilizing such cell lines are very well known in the art. For example, sequences encoding canine PPARγ can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

As those skilled in the art will appreciate, based upon the present disclosure, any number of selection systems may be used to recover transformed cell lines. These include, but are not limited to, the herpes simplex virus thymidine kinase and adenine phosphoribosyltransferase genes, for use in tk- and apr-cells, respectively (see, e.g., Wigler, M. et al. (1977) Cell 11, 223-232; Lowy, I. et al. (1980) Cell 22, 817-823). Antimetabolite, antibiotic, or herbicide resistance can also be used as a basis for selection. For example, dhfr (dihydrofolate reductase) confers resistance to methotrexate; neo confers resistance to the aminoglycosides neomycin and G-418; and als and pat confer resistance to chlorsulfuron and phosphinotricin acetyltransferase, respectively (see, Wigler, M. et al. (1980) Proc. Natl. Acad. Sci. USA 77, 3567-3570; Colbere-Garapin, F. et al. (1981) J. Mol. Biol. 150, 1-14). Additional selectable genes have been described, e.g., trpB and hisD, which alter cellular requirements for metabolites (see, e.g., Hartman, S. C., et al. (1988) Proc. Natl. Acad. Sci. USA 85, 8047-8051). Visible markers, e.g., anthocyanins, green fluorescent proteins (Clontech, Palo Alto, Calif.), β glucuronidase and its substrate β-glucuronide, or a luciferase and its substrate luciferin may be used. These markers can be used not only to identify transformants, but also to quantify the amount of transient or stable protein expression attributable to a specific vector system (see, e.g., Rhodes, C. A. (1995) Methods Mol. Biol. 55,121-131).

Although the presence/absence of marker gene expression suggests that the gene of interest is also present, the presence and expression of the gene may need to be confirmed. For example, if the sequence encoding canine PPARγ is inserted within a marker gene sequence, transformed cells containing sequences encoding canine PPARγ can be identified by the absence of marker gene function. Alternatively, a marker gene can be placed in tandem with a sequence encoding canine PPARγ under the control of a single promoter. As those skilled in the art will appreciate, based upon the present disclosure, expression of the marker gene in response to induction or selection generally indicates expression of the tandem gene as well.

Host cells that contain a nucleotide sequence encoding a canine PPARγ and that express the canine PPARγ may be identified by a variety of procedures known to those of skill in the art based upon the present disclosure. These procedures include, but are not limited to, DNA-DNA or DNA-RNA hybridizations, PCR amplification, and protein bioassay or immunoassay techniques which include membrane-, solution- and/or chip-based technologies for the detection and/or quantification of nucleotide or amino acid sequences.

Host cells transformed with nucleotide sequences encoding canine PPARγ polypeptide, based upon the present description, may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode a canine PPARγ polypeptide may be designed to contain, for example, signal sequences, which direct secretion of canine PPARγ through a prokaryotic or eukaryotic cell membrane.

It will be appreciated by those skilled in the art based upon the present description, that a host cell strain may also be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used, for example, to specify protein targeting, folding, and/or activity. Different host cells which have specific cellular machinery and characteristic mechanisms for post-translational activities (e.g., CHO, HeLa, MDCK, HEK293, and W138) are available from the ATCC and may be chosen to ensure the desired modification and processing of the foreign protein.

In another embodiment of the invention, immunological methods are used for detecting and measuring the expression of canine PPARγ. For example, such methods include using specific polyclonal or monoclonal antibodies. Such methods are well known in the art. Examples of such techniques include enzyme-linked immunosorbent assays (ELISAs), radioimmunoassays (RIAs), and fluorescence activated cell sorting (FACS). These and other assays are well known in the art (see, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York, N.Y.; and Pound, J. D. (1998) Immunochemical Protocols, Humana Press, Totowa N.J.).

In a further embodiment, a wide variety of labels and conjugation techniques may be used in various nucleic acid and amino acid assays. Such techniques are known by those skilled in the art. Means for producing labeled hybridization or PCR probes for detecting sequences related to polynucleotides encoding canine PPARγ include oligolabeling, nick translation, end-labeling, or PCR amplification using a labeled nucleotide.

Alternatively, the sequences encoding canine PPARγ, or any fragments thereof, may be cloned into expression vectors for the production of a messenger RNA (mRNA) probe. Such expression vectors are known in the art and are commercially available. The expression vectors, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech (Piscataway, N.J.), Promega (Madison, Wis.), and US Biochemical (Cleveland, Ohio). Suitable reporter molecules or labels which may be used for ease of detection in the assays of the invention include, for example, radionuclides, enzymes, fluorescent, chemiluminescent, or chromogenic agents, as well as substrates, cofactors, inhibitors, magnetic particles, and the like.

Methods for preparing and using probes and primers are described in the references, for example Sambrook et al., 1989, Molecular Cloning: A Laboratory Manual, 2nd ed., vol. 1-3, Cold Spring Harbor Press, Plainview N.Y.; Ausubel et al.,1987, Current Protocols in Molecular Biology, Greene Pub;. Assoc. & Wiley-Intersciences, New York, N.Y.; Innis et al., 1990, PCR Protocols, A Guide to Methods and Applications, Academic Press, San Diego, Calif. PCR primer pairs can be derived from a known sequence, for example, by using computer programs intended for that purpose such as Primer (Version 0.5, 1991, Whitehead Institute for Biomedical Research, Cambridge Mass.).

In another embodiment of the invention, natural, modified, or recombinant nucleic acid sequences encoding canine PPARγ may be ligated to a heterologous sequence resulting in translation of a fusion protein in any suitable host systems, including those described above. For example, a chimeric canine PPARγ protein containing a heterologous moiety that can be recognized by a commercially available antibody may facilitate the screening of peptide libraries for inhibitors of canine PPARγ activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the canine PPARγ encoding sequence and the heterologous protein sequence, so that canine PPARγ may be cleaved away from the heterologous moiety following purification. Methods for fusion protein expression and purification are discussed, for example, in Ausubel (1995, supra, ch. 10). Those skilled in the art will appreciate, based on the present description, that a variety of commercially available kits may also be used to facilitate expression and purification of fusion proteins of the invention.

In a further embodiment of the invention, synthesis of radiolabeled canine PPARγ may be achieved in vitro using the TNT rabbit reticulocyte lysate or wheat germ extract system (Promega, Madison, Wis.). These systems couple transcription and translation of protein-coding sequences operably associated with the T7, T3, or SP6 promoters. Translation takes place in the presence of a radiolabeled amino acid precursor, for example, ³⁵S-methionine.

Fragments of canine PPARγ may be produced not only by recombinant means, but also by direct peptide synthesis using solid-phase techniques (see, e.g., Creighton, supra, pp. 55-60). Protein synthesis may be performed by manual techniques or by automation. Automated synthesis may be achieved, for example, using the ABI 431A peptide synthesizer (Perkin-Elmer). Various fragments of canine PPARγ may be synthesized separately and then combined to produce the full-length molecule.

In another aspect of this invention, the nucleotide sequences encoding canine PPARγ may be used for diagnostic purposes. The nucleotide sequences which may be used include oligonucleotide sequences, complementary RNA and DNA molecules, and peptide nucleic acid (PNA) molecules. PNAs are antisense molecules or anti-gene agents which comprise an oligonucleotide linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell. The polynucleotides may be used to detect and quantify gene expression in biopsied tissues in which expression of canine PPARγ may be correlated with disease. The diagnostic assays may be used to determine absence, presence, and excess expression of canine PPARγ, and to monitor regulation of canine PPARγ levels during therapeutic intervention.

In the diagnostic aspects, hybridization with PCR probes which are capable of detecting polynucleotide sequences, including genomic sequences, encoding canine PPARγ or closely related molecules may be used to identify nucleotide sequences which encode canine PPARγ. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding canine PPARγ, allelic variants, or related sequences. Means for producing specific hybridization probes for DNAs encoding canine PPARγ include, for example, the cloning of nucleotide sequences encoding canine PPARγ or canine PPARγ derivatives into vectors for the production of mRNA probes. Expression vectors that can be for insertion of a nucleotide sequences encoding canine PPARγ are known in the art, are commercially available, and may be used to synthesize RNA probes in vitro by means of the addition of the appropriate RNA polymerases and the appropriate labeled nucleotides. Hybridization probes may be labeled by any suitable reporter group, for example, by radionuclides such as ³²P or ³⁵S, or by enzymatic labels, such as alkaline phosphatase coupled to the probe via avidin/biotin coupling systems, and the like.

In another aspect of the invention, nucleotide sequences encoding canine PPARγ may be used for the diagnosis of disorders associated with expression of canine PPARγ. The polynucleotide sequences encoding canine PPARγ may be used in Southern or northern analysis, dot blot, or other membrane-based technologies; in PCR technologies; in dipstick, pin, and multiformat ELISA-like assays; and in microarrays utilizing fluids or tissues from patients to detect altered canine PPARγ expression. Such qualitative or quantitative methods are well known in the art.

In one embodiment of the invention, the nucleotide sequences encoding canine PPARγ are useful in assays that detect the presence of disorders associated with expression of canine PPARγ. The nucleotide sequences encoding. canine PPARγ are labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample, then the presence of altered levels of nucleotide sequences encoding canine PPARγ in the sample indicates the presence of the associated disorder. Those skilled in the art will appreciate, based upon the present description, that such assays may also be used to evaluate the efficacy of a particular therapeutic treatment regimen in animal studies, in clinical trials, and/or to monitor the treatment of an individual patient.

To provide a basis for the diagnosis of a disorder associated with expression of canine PPARγ, a normal or standard profile for expression is established for a particular type of subject. This may be accomplished by combining body fluids or cell extracts taken from normal subjects (i.e., those who are substantially free of the disorder), with a sequence, or a fragment thereof, encoding canine PPARγ, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Statistically significant deviations from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

Methods which may also be used to quantify the expression of canine PPARγ include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer of interest is presented in various dilutions and a spectrophotometric or colorimetric response gives rapid quantitation.

In further embodiments, oligonucleotides or longer fragments derived from any of the polynucleotide sequences described herein are used as targets in a microarray. As those skilled in the art will appreciate, based upon the present description, the microarray can be used to monitor the expression level of large numbers of genes simultaneously and to identify genetic variants, mutations, and polymorphisms. This information may be used to determine, for example, gene function, to understand the genetic basis of a disorder, to diagnose a disorder, and to develop and monitor the activities of therapeutic agents.

Microarrays may be prepared, used, and analyzed using methods known in the art (see, e.g., U.S. Pat. No. 5,474,796; Schena, M. et al. (1996) Proc. Natl. Acad. Sci. USA 93,10614-10619; PCT International Application Publication WO95/251116; PCT International Application Publication WO95/35505; Heller, R. A. et al. (1997) Proc. Natl. Acad. Sci. USA 94, 2150-2155; and U.S. Pat. No. 5,605,662).

A chemical coupling procedure and an ink jet device can be used to synthesize array elements on the surface of a substrate (see, e.g., Baldeschweiler, supra). An array analogous to a dot or slot blot may also be used to arrange and link elements to the surface of a substrate using thermal, UV, chemical, or mechanical bonding procedures. A typical array may be produced by hand or using available methods and machines and contain any appropriate number of elements. After hybridization, nonhybridized probes are removed and a scanner used to determine the levels and patterns of fluorescence. The degree of complementarity and the relative abundance of each probe which hybridizes to an element on the microarray may be assessed through analysis of the scanned images.

Full-length cDNAs, expressed sequence tags (ESTs), or fragments thereof may comprise the elements of the microarray. Fragments suitable for hybridization can be selected using software well known in the art such as LASERGENE software (DNASTAR). Full-length cDNAs, ESTs, or fragments thereof corresponding to one of the nucleotide sequences of the present invention, or selected at random from a cDNA library relevant to the present invention, are arranged on an appropriate substrate, e.g., a glass slide. The cDNA is fixed to the slide using, e.g., ultra-violet cross-linking followed by thermal and chemical treatments and subsequent drying (see, e.g., Schena, M. et al. (1995) Science 270, 467-470; Shalon, D. et al. (1996) Genome Res. 6, 639-645). Fluorescent probes are prepared and used for hybridization to the elements on the substrate. The substrate is analyzed by procedures well known in the art, for example, by scanning and analyzing images of a microarray.

In another embodiment of the invention, nucleotide sequences encoding canine PPARγ are used to generate hybridization probes useful in mapping the naturally occurring genomic sequence. The sequences may be mapped, for example, to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries, as desired (see, e.g., Harrington, J. J. et al. (1997) Nat. Genet. 15, 345-355; Price, C. M. (1993) Blood Rev. 7, 127-134; and Trask, B. J. (1991) Trends Genet. 7, 149-154).

Fluorescent in situ hybridization (FISH) may be correlated with other physical chromosome mapping techniques and genetic map data. (see, e.g., Heinz-Ulrich, et al. (1995) in Meyers, supra, pp. 965-968). Examples of genetic map data can be found in various scientific journals or at the Online Mendelian Inheritance in Man (OMIM) online internet website. Correlation between the location of the gene encoding canine PPARγ on a physical chromosomal map and a specific disorder, or a predisposition to a specific disorder, may help define the region of DNA associated with that disorder. The nucleotide sequences of the invention may be used to detect differences in gene sequences among normal, carrier, and affected individuals.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for disease genes using positional cloning or other gene discovery techniques. Once the disease or syndrome has been crudely localized by genetic linkage to a particular genomic region, e.g., ataxia-telangiectasia to 11 q22-23, any sequences mapping to that area may represent associated or regulatory genes for further investigation (see, e.g., Gatti, R. A. et al. (1988) Nature 336:577-580). The nucleotide sequences of the subject invention may also be used to detect differences in the chromosomal location due, for example, to translocation, inversion, etc., among normal, carrier, or affected individuals.

In another embodiment of the invention, canine PPARγ, its catalytic or immunogenic fragments, or oligopeptides thereof, can be used for screening agents, for example, screening libraries of compounds in any of a variety of drug screening techniques. The fragment employed in such screening may be, for example, free in solution, affixed to a solid support, borne on a cell surface, or located intracellularly. As those skilled in the art will appreciate, based upon the present description, the formation of binding complexes between canine PPARγ and the agent being tested may be measured. Such agents may include small molecule compounds, peptides, lipids, etc. or combinations thereof.

Another technique for drug screening provides for high throughput screening of compounds having suitable binding affinity to the protein of interest (see, International Patent Application Publication No. WO84/03564). In this method, large numbers of different small test compounds are synthesized on a solid substrate. The test compounds are reacted with canine PPARγ, or fragments thereof, and washed. Bound canine PPARγ is then detected by methods well known in the art, based upon the present invention. Purified canine PPARγ can also be coated directly onto plates for use in drug screening techniques, including the those described above. Alternatively, non-neutralizing antibodies can be used to capture the peptide and immobilize it on a solid support.

In another embodiment, competitive drug screening assays may be used in which neutralizing antibodies capable of binding canine PPARγ specifically compete with a test agent or compound for binding canine PPARγ. In this manner, as those skilled in the art will appreciate, based upon the present description, antibodies can be used to detect the presence of any peptide which shares one or more antigenic determinants with canine PPARγ.

In additional embodiments, the nucleotide sequences which encode canine PPARγ may be used in any molecular biology techniques that have yet to be developed, provided the new techniques rely on properties of nucleotide sequences that are currently known, including, but not limited to, such properties as the triplet genetic code and specific base pair interactions.

This invention is also based, in part, on the discovery that canine PPARγ is expressed in canine cancer cells. It has been reported that an agonist of PPARγ can induce human cancer cells to terminally differentiate (see, Demetri, et al., (1999) Proc. Natl. Acad. Sci. USA 96, 3951-3956). As a result of this differentiation, a PPARγ would serve as a cancer treatment. It has now been discovered that PPARγ is expressed in canine cancer cells and that an activator of PPARγ may be used as a therapy for canine cancer. The expression of canine PPARγ is illustrated in the Examples provided below.

The present invention is further based, in part, on the discovery that an activator of canine PPARγ reduces the level of expression of the cytokine, tumor necrosis factor alpha (TNF-α), in canine cells. Notably, it was discovered that when canine cells are treated with the TNF-A stimulating compound, phorbol 12-myristate 13-acetate (PMA), a PPARγ agonist is able to reduce TNF-α expression in the stimulated cells. The suppression of cytokine release, especially that of TNF-α, is important in the treatment of allergic and inflammatory diseases (see Queralt, et al., (2000) Inflamm. Res. 49, 355-360). Thus, a PPARγ activator may be used to treat diseases or conditions related to increased TNF-α expression and/or to beneficially reduce TNF-α expression. The effect of PPARγ activators TNF-α expression in canine cells is illustrated in the Examples provided below.

As those skilled in the art will appreciate, based upon the present description, any PPARγ activator may be used in the practice of the therapeutic method aspects of this invention. Exemplary PPARγ activators include those described in the following U.S. Patents and publications: U.S. Pat. No. 4,340,605; U.S. Pat. No. 4,342,771; U.S. Pat. No. 4,367,234; U.S. Pat. No. 4,617,312; U.S. Pat. No. 4,687,777; U.S. Pat. No. 4,703,052; Shibata, et al., (1999) Eur J Pharmacol 364, 211-19; Cobb, et al., (1998) J Med Chem 41, 5055-69; Suh, et al., (1999) Cancer Res 59, 5671-3; Li, et al., (2000) J Clin Invest 106, 523-31; and Henke, et al., (1999) Bioorg Med Chem Lett 9, 3329-34; and analogs, derivatives, prodrugs and pharmaceutically acceptable salts thereof. In the practice of the invention, preferred PPARγ activators include darglitazone, troglitazone, ciglitazone, englitazone, pioglitazone, also known as Actos®, and rosiglitazone, also known as Avandia®, and BRL-49653. PPARγ activators are preferably administered in amounts ranging from about 0.1 mg/day to about 100 mg/day in single or divided doses, preferably about 0.1 mg/day to about 50 mg/day for an average subject, depending upon the PPARγ activator and the route of administration. However, some variation in dosage will necessarily occur depending on the condition of the canine subject being treated. The individual responsible for dosing will, in any event, determine the appropriate dose for the individual subject, based on the present description.

In the therapeutic method aspects of this invention, a PPARγ activator may be administered alone or with one or more pharmaceutically acceptable carriers, diluents or vehicles. Pharmaceutical compositions containing a PPARγ activator may be readily administered in any suitable dosage forms such as tablets, powders, syrups, injectable solutions and so forth. These pharmaceutical compositions may, if so desired, contain additional ingredients such as flavorings, binders, excipients and the like. For the purposes of oral administration, tablets containing various excipients such as sodium citrate, calcium carbonate, and calcium diphosphate may be used along with various disintegrants such as starch, alginic acid and certain complex silicates, together with binding agents such as polyvinylpyrrolidone, sucrose, gelatin and acacia. Additionally, lubricating agents such as magnesium stearate, sodium lauryl sulfate, and talc are often useful for tabletting purposes. Solid compositions of a similar type may also be used as fillers in soft and hard filled gelatin capsules. Preferred materials for this use include lactose or milk sugar and high molecular weight polyethylene glycols. When aqueous suspensions or elixirs are desired for oral administration, the PPARγ activator therein may be combined with various sweetening or flavoring agents, coloring matter or dyes and, if desired, emulsifying or suspending agents, together with diluents such as water, ethanol, propylene glycol, glycerin or various combinations thereof, as suitable, based upon the present description.

For parenteral administration, solutions of a PPARγ activator useful in this invention, include for example, solutions in sesame or peanut oil or aqueous propylene glycol, or sterile aqueous solution may be employed. Such aqueous solutions should be suitably buffered if necessary and the liquid diluent first rendered isotonic, for example, with sufficient saline and/or glucose. These particular aqueous solutions are especially suitable for intravenous, intramuscular, subcutaneous and intraperitoneal administration. In this connection, the sterile aqueous media employed are all readily available by standard techniques known to those skilled in the art.

The disclosures of all patents, applications, publications and documents, for example brochures or technical bulletins, cited herein, are hereby expressly incorporated by reference in their entirety.

Without further elaboration, it is believed that one skilled in the art can, using the present description, including the examples, drawings, sequence listings and attendant claims, utilize the present invention to its fullest extent. The following embodiments are, therefore, to be construed as merely illustrative and not limitative of the remainder of the disclosure in any manner whatsoever.

### EXAMPLES

### Example 1

### Preparation of a Polynucleotide Encoding a Canine PPARγ from Canine Adipose Tissue

Total mRNA was isolated from canine adipose tissue. cDNA encoding canine PPARγ was synthesized from the mRNA by RT-PCR using ATGGGTGAAACTCTGGGAGA as the 5′ primer and CTAGTACAAGTCCTTGTA as the 3′ primer, resulting in a 1.5 Kb canine PPARγ polynucleotide. The PCR reactions were carried out for 35 cycles as follows: denaturation for two minutes at 94° C.; annealing for one minute at 52° C. and extension for seven minutes at 72° C.

### Example 2

### Expression of PPARγ in Canine Adipose Tissue and in Canine Cancer Cells

Western blots were prepared using the human polyclonal PPARγ antibody (ABR #PA3-821, Affinity BioReagents, Golden, Colo.). Canine adipose tissue was used as a positive control. The following cell lines were used to prepare the blots:

| | |
|---|---|
| Cell Line Designation: | CF21.T |
| ATCC Number: | CRL-6220 |
| Tissue: | canine cancer, connective tissue |
| Cell Line Designation: | A-72 |
| ATCC Number: | CRL-6243 |
| Tissue: | canine mammary tumor |
| Cell Line Designation: | 3T3-L1 |
| ATCC Number: | CL-173 (CCL-92.1) |
| Tissue: | mouse embryo adipocytes |
| Contributor: | Mass. Inst. Tech. |
| Cell Line Designation: | A-72 |
| ATCC Number: | CRL-1542 |
| Tissue: | canine, unknown tumor |
| Contributor: | L. N. Binn |
| Cell Line Designation: | D-17 |
| ATCC Number: | CCL-183 |
| Tissue: | canine osteosarcoma |
| Contributor: | W. A. Nelson-Rees |

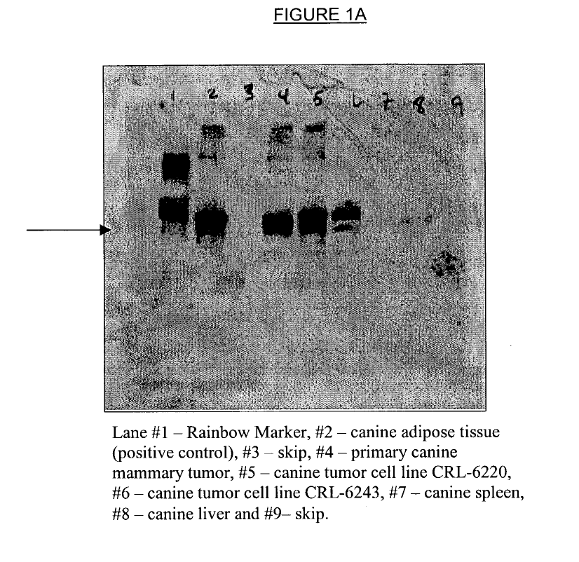

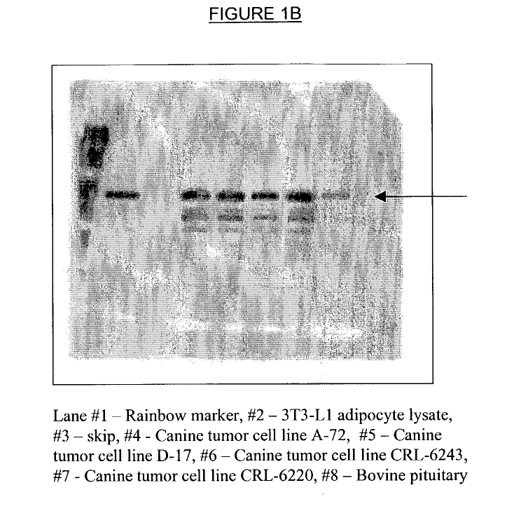

PPARγ was found to be expressed in canine adipose tissue, canine primary mammary tumor and in four canine tumor cell lines. These results are illustrated in the Western blots of FIGS. 1A and 1B.

### Example 3

### Effect of PPARY Agonist on TNF-α mRNA Levels

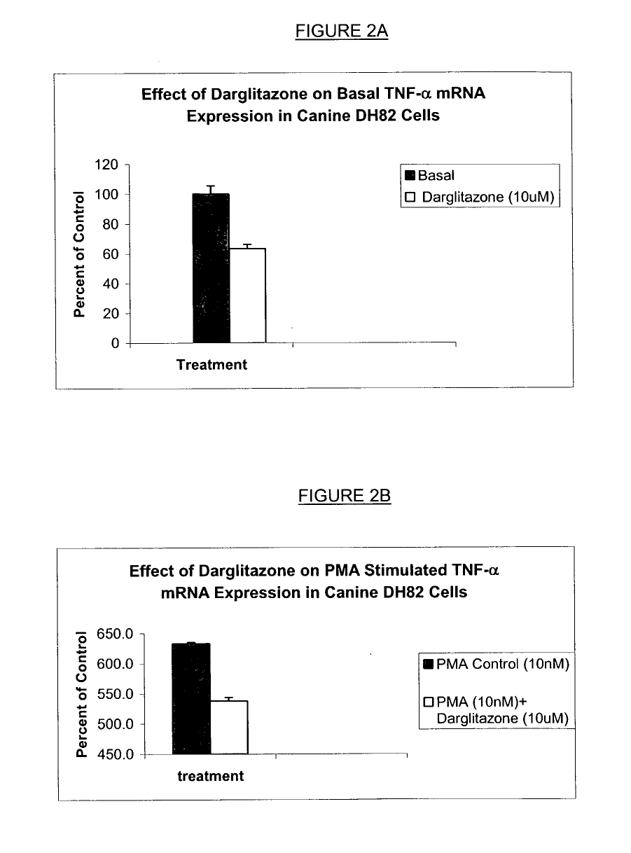

Canine DH82 cells (ATCC No. CRL-10389) were cultured in RPMI 1640 medium containing 2 mM L-glutamine, 25 mM Hepes, 10% fetal bovine serum (FBS) and 1× antimycotic at 37° C., 95% humidity, and 5% CO₂. Cells were treated with 10 nM PMA (Sigma-Aldrich, St. Louis, Mo.), PMA (10 nM) plus darglitazone (10 uM), and darglitazone (10 μM), respectively for two hours. After treatment, cell RNA lysis buffer was added to the cells. Samples were loaded into MagNa Pure™ machine (F. Hoffmann-La Roche, Basel, Switzerland). MagNa Pure LC™ RNA isolation Kit II (F. Hoffmann-La Roche) was used for isolation of total RNA from culture cells. First strand cDNA synthesis kit for RT-PCR (F. Hoffmann-La Roche) was used to synthesize cDNA. Samples were incubated at 25° C. for 10 minutes and then at 42° C. for 60 minutes, denatured at 90° C. for 5 minutes and cooled to 4° C. for 1-2 hr or at −20° C. for longer periods. Quantification PCR (Q-PCR) was performed using the Lightcycler Faster DNA Master SYBR Green 1™ kit (F. Hoffmann-La Roche) using canine TNF-α primers (forward primer: 5′ CCMGTGACMGCCAGTAGCTC, reverse primer: 5′ ATGAGGTACAACCCATCTGACG). Canine GAPDH primers were used to amplify the GAPDH gene in order to normalize the TNF-α gene expression (GAPDH forward primer: 5′ TTTGTGATGGGCGTGAAC, and reverse primer: 5′ ATGGACGGTGGTCATGAG). The PCR cycle was carried out 35 times. Each cycle includes: denaturing for 10 minutes at 95° C. followed by one second or less at 99° C.; annealing for 10 seconds at 60° C.; and extension for 20 seconds at 72° C. For quantification, melting points were measured every 0.2° C. from 60° C. to 99° C. using fluorescence detection. The reaction stopped by cooling down to 30° C. These results are illustrated in the Western blots of FIGS. 2A and 2B.

As shown by FIGS. 2A and 2B, the commercially available PPARγ agonist, darglitazone, was found to reduce basal TNF-α expression in canine DH82 cells. More notably, when canine cells where treated with the TNF-α stimulating compound, phorbol 12-myristate 13-acetate (PMA), darglitazone reduce TNF-A expression in the stimulated cells. FIG. 2A compares the effect of darglitazone to basal TNF-A levels in the canine cell line, DH82. FIG. 1B shows the effect of darglitazone on PMA stimulated DH82 cells. The height of the columns in FIGS. 2A and 2B correspond to the percent of TNF-α expression relative to the expression level in the control (basal level of TNF-α expression—left column of FIG. 2A), wherein the control is set to 100%. Accordingly, FIG. 2A shows that darglitazone reduces TNF-α expression by about 40% from the basal level. FIG. 2B shows that in PMA stimulated cells, darglitazone reduces TNF-A expression by about 15%.

1. An isolated polynucleotide encoding a canine PPARγ polypeptide.

2. An isolated polynucleotide comprising a polynucleotide sequence that encodes a polypeptide sequence of SEQ ID NO: 2.

3. An isolated polynucleotide comprising a polynucleotide sequence that encodes a polypeptide sequence having at least about 98% amino acid identity with the polypeptide sequence that is encoded by the polynucleotide sequence of SEQ ID NO: 1.

4. An isolated polynucleotide characterized as hybridizing under high stringency conditions with a polynucleotide sequence of SEQ ID NO: 1.

5. An isolated polynucleotide comprising a polynucleotide sequence having at least about 92% nucleotide identity with the polynucleotide sequence of SEQ ID NO: 1.

6. An isolated polynucleotide of claim 5 wherein said polynucleotide sequence has at least about 95% nucleotide identity with the polynucleotide sequence of SEQ ID NO: 1.

7. An isolated polynucleotide of claim 6 wherein said polynucleotide sequence is the polynucleotide sequence of SEQ ID NO: 1.

8. An isolated canine PPARγ polypeptide.

9. An isolated polypeptide comprising a polypeptide sequence having at least about 98% amino acid identity with the polypeptide sequence that is encoded by the polynucleotide sequence of SEQ ID NO: 1.

10. An isolated polypeptide of claim 9 wherein said polypeptide sequence comprises a polypeptide sequence that is encoded by the polynucleotide sequence of SEQ ID NO: 1.

11. An expression vector comprising a polynucleotide encoding a canine PPARγ polypeptide.

12. An expression vector comprising a polynucleotide sequence that encodes a polypeptide sequence of SEQ ID NO: 2.

13. An expression vector comprising a polynucleotide sequence having at least about 92% nucleotide identity with the polynucleotide sequence of SEQ ID NO: 1.

14. An expression vector of claim 13 wherein said polynucleotide sequence has at least about 95% nucleotide identity with the polynucleotide sequence of SEQ ID NO: 1.

15. An expression vector of claim 14 wherein said polynucleotide sequence comprises the polynucleotide sequence of SEQ ID NO: 1.

16. An expression vector of claim 11 as deposited with the ATCC having ATCC deposit number UC-25463.

17. A transformed cell comprising an inserted polynucleotide comprising a polynucleotide sequence that encodes a canine PPARγ polypeptide, or a progeny cell thereof comprising said polynucleotide sequence.

18. A transformed cell comprising an inserted polynucleotide comprising a polynucleotide sequence that encodes a polypeptide sequence of SEQ ID NO: 2, or a progeny cell thereof comprising said polynucleotide sequence.

19. A transformed cell comprising an inserted polynucleotide comprising a polynucleotide sequence having at least about 92% nucleotide identity with the polynucleotide sequence of SEQ ID NO: 1, or a progeny cell thereof comprising said polynucleotide sequence.

20. A transformed cell comprising an inserted polynucleotide comprises a polynucleotide sequence having at least about 95% nucleotide identity with the polynucleotide sequence of SEQ ID NO: 1, or a progeny cell thereof comprising said polynucleotide sequence.

21. A transformed cell comprising an inserted polynucleotide comprises a polynucleotide sequence of SEQ ID NO: 1, or a progeny cell thereof comprising said polynucleotide sequence.

22. An isolated antibody which specifically binds to a polypeptide encoded by the sequence of SEQ ID NO: 1.

23. A microarray comprising an isolated polynucleotide encoding a canine PPARγ polypeptide, or a complimentary nucleotide sequence thereof.

24. A microarray of claim 23 wherein said polynucleotide sequence is the polynucleotide sequence of SEQ ID NO: 1, or a complimentary nucleotide sequence thereof.

25. A microarray comprising a nucleotide sequence comprising at least 25 contiguous nucleotides of the nucleotide sequence of SEQ ID NO: 1, or a complimentary nucleotide sequence thereof.

26. A method comprising administering a PPARγ activating amount of a PPARγ activator to a mammal of the Canidae family, preferably a member of the species, Canis lupus familiaris, having a disease, condition or disorder which is beneficially treated by an activator of canine PPARγ.

27. A method comprising administering a PPARγ inhibiting amount of a PPARγ activator to a mammal of the Canidae family, preferably a member of the species, Canis lupus familiaris, having a disease, condition or disorder which is beneficially treated by an activator of canine PPARγ.

28. A method comprising administering a PPARγ activating amount of a PPARγ activator to a mammal of the Canidae family having a disease or condition selected from:
a disease or condition characterized by the cellular expression of TNF-α that is higher than normal;
cancer; and
diabetes mellitus.

29. A method of claim 28 wherein said disease or condition is a disease or condition characterized by the cellular expression of TNF-α that is higher than normal, selected from inflammation, allergy, dermatitis, arthritis, psoriasis, inflammatory bowel disease and endotoxemia.

30. A method of claim 28 wherein said PPARγ activator is selected from darglitazone, troglitazone, ciglitazone, englitazone, pioglitazone and rosiglitazone.

31. A method of claim 28 wherein said mammal is a member of the species, Canis lupus familiaris.


SEQUENCE LISTING (2 sequences)

SEQ ID NO: 1 - DNA, 1518 bp, Canine
atgggtgaaactctgggagattctctcattgacccagaaagcggctcctttgctgatacactgtctgcaagcacttcacaagaaactaccatggttgacacagagatgccattctggcccaccaactttggaatcagttcagtggatctgtccgtaatggatgaccactcccattcttttgacatcaagccattcaccaccgtggatttctccagcatttccactccacactatgaagacattccattctcaagagcggacccgatggttgcagattataagtatgatctgaagctccaagagtatcaaagtgcaatcaaagtggagcctgcatccccaccttattattctgaaaagactcagctgtacaataagcctcacgaagagccttccaactccctcatggcaattgaatgccgagtctgtggagataaagcttctgggttccactatggagttcatgcttgtgaaggatgcaagggcttcttccggaggacaatcagattgaaactcatttacgatagatgtgacctaaactgtcggatccacaaaaaaagtagaaataaatgtcagtactgtcggtttcagaagtgccttgctgtggggatgtctcataatgccatcaggtttgggcggatgccacaggctgagaaggaaaagctgttggcggagatctccagtgatatcgaccagctgaacccagagtcggctgatctccgggccctggcaaaacatttgtatgactcatacataaagtccttcccgctgaccaaagcaaaggcgagagcgatcttgacaggaaagacaacagacaaatcaccatttgttatctatgacatgaattccttaatgatgggagaagataaaatcaagttcaaacacatcacccccttgcaggagcagaacaaagaggtggccattcgcatctttcaggggtgtcagttccgctcagtggaagccgtgcaggagatcacagagtacgccaaaagtatccctggttttgtaaaccttgacttgaatgaccaagtaactctgctaaaatatggtgtccatgagatcatttacacgatgctggcgtccttgatgaataaagacggggttctcatatcagaaggccaaggattcatgacaagggagtttctaaagagcctgagaaagccctttggtgacttcatggagccaaagtttgagtttgctgtgaagttcaatgcactggaattagatgacagcgacttggcaatatttatagccgtcattattctcagtggagaccgcccaggtttgctgaatgtgaagcccattgaggacatacaggacaacctgctacaagccttggagctccagctcaagctgaaccaccccgagtcctctcagctctttgccaagctgctccagaaaatgacagacctcagacaaattgtaacagaacatgtgcagctattgcaagtcataaagaaaacagagacagacatgagtcttcacccactcctacaagaaatatacaaggacttgtactag

SEQ ID NO: 2 - protein, 505 aa, Canine
MGETLGDSLIDPESGSFADTLSASTSQETTMVDTEMPFWPTNFGISSVDLSVMDDHSHSFDIKPFTTVDFSSISTPHYEDIPFSRADPMVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQNKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY